(19) United States
(12) Patent Application Publication
(10) Pub. No.: US 2023/0290523 A1
(43) Pub. Date: Sep. 14, 2023

(54) SYSTEM AND METHOD FOR HEALTHCARE MANAGEMENT

(71) Applicants: Dwayne Hall, View Park, CA (US); Akwi Devine, View Park, CA (US)

(72) Inventors: Dwayne Hall, View Park, CA (US); Akwi Devine, View Park, CA (US)

(21) Appl. No.: 18/105,179

(22) Filed: Feb. 2, 2023

Related U.S. Application Data
(60) Provisional application No. 63/306,214, filed on Feb. 3, 2022.
(60) Provisional application No. 63/391,223, filed on Jul. 21, 2022.

(51) Int. Cl. G16H 80/00; G16H 50/20; G16H 50/70; G16H 50/30; G16H 10/60

(52) U.S. Cl. CPC G16H 80/00; G16H 50/20; G16H 50/70; G16H 50/30; G16H 10/60

(57) ABSTRACT

The present invention is a platform providing value for two customer segments: patients and health professionals. The platform combines user friendly functionality for holistic and western medicine practitioners and facilitates engagement across American Medical Association (“AMA”) and non-AMA recognized fields of specialty. The platform manages, facilitates, and supports both episodic and urgent care support for cash pay, insurance pay, and low-income or subsidized patients allowing for broadened access to care within a vast array of communities and regions thus expanding, expediting, and ensuring immediate access to medical care and treatment through a virtual and non-virtual means.

### CROSS REFERENCE TO RELATED APPLICATIONS

This application claims the benefit of U.S. Provisional Patent Application Ser. No. 63/306,214, filed Feb. 3, 2022, and U.S. Provisional Patent Application Ser. No. 63/391,223, filed Jul. 21, 2022, the contents of which are incorporated herein by reference.

### BACKGROUND OF THE INVENTION

The present invention is directed to healthcare management.

Existing platforms that are providing virtual care are driving value through arranging visits or virtual consultations. Patient engagement is limited, and patient outcomes are impacted because the focus is on urgent care and not prevention, holistic, coordinated, or primary care. Existing platforms can increase virtual care costs, extend wait times, operate with the non-use of certified professional nurses and providers, and limit support for the patient's long term health needs. These existing platforms fail to tap into stratified markets (of both market rate payers and low-income persons), and fail to offset physician and other healthcare provider costs by building in sub-licensing structures to improve the profitability of physician and other health providers practices and help in the establishment and growth of virtual practices due to the primary care capabilities. As well, existing platforms fail to adequately address inherent biases within healthcare.

Certain community initiatives and groups are striving to provide consistent care that is comprehensive, patient-centered, and addresses both the health and social needs of patients experiencing episodic itinerancy, transitional displacement, transient living situations and economic challenges. Ultimate success will be moving the patients of this population from urgent care to preventative care. Additionally, the platform uses advanced tools to not only track wellness, but projects life span based upon the present state of health of the patient. Therefore, outcomes may be managed towards lengthening life span through intervention.

These initiatives aim to create a population health system to coordinate street medicine services amongst all providers in large population centers to ensure linkages to specialty and direct care, medical homes, housing, and other supportive services. There is an urgent demand for a web and mobile-friendly software solution platform to allow for timely bi-directional data exchange, patient care management, staff management, and secure communication across existing software solutions.

### SUMMARY OF THE INVENTION

The present invention pertains to a system and method for a multi-functional platform that provides medical practitioners with a turnkey solution by way of a technologically advanced tool, in compliance with the Health Insurance Portability and Accountability Act (“HIPAA”) that creates a concierge and patient centric experience for all patients. The platform can integrate with external platforms to facilitate physicians and other health care team members to establish, manage, expand, and market independent physician practices, or independent physicians without a practice group, to an unlimited number of patients in a HIPAA compliant and organized manner, while reducing overhead by negating the need for office space, permanent staff, or costly infrastructure.

The platform functions as a system and method of delivering care through a virtualized means with advanced tools of managing, treating, and coordinating care around educating, coaching, and sustaining primary care. The platform incorporates advanced technology, such as graphical and artificial intelligence (“AI”) tools, allowing for the patient to customize care as their needs and those of their family are used to create services that are anticipatory of their unique health conditions as they age or experience changes in their health condition. The platform also provides for caregiver collaboration and facilitates access to PHI to support the coordination of care.

The present invention's platform is capable of deployment into underserved and rural regions to expand the availability of basic and advanced care, as well as allowing for lower income communities to access qualified physicians by matching low-income persons to physicians and other health care providers offering pro-bono care and support. The platform decreases the burdens on the emergency rooms and lowers overall per patient healthcare costs by focusing on prevention, supporting sustainable outcomes by reinforcing and using holistic care plans, and expanding job creation and employment opportunities for health professionals and independent physician practices. The platform can be used as a turnkey revenue producing tool that enables concierge care, in office and virtual staff expansion and management, and a HIPAA compliant means of delivering services to patients.

The platform is able to provide health care access to “care deserts” wherein limited access to medical care increases instances of aggravated illness and taxes the public care system. The virtual platform allows physicians and other health care team members to provide voluntary pro-bono care to individuals experiencing displacement, housing insecurity, transitional or vagrant living situations from economic crises, as well as those who live under itinerant conditions and low-income communities (“LICs”). The platform incorporates the use of the US Treasury Community Development Financial Institution (“CDFI”) mapping system to identify, track, and measure engagement within LICs with health professionals.

The present invention is a customized external Application Programming Interface (“API”) platform that integrates patient data, health care resources and information through mobile online accessibility, search, geolocation, and notification functionality. The API platform facilitates health care professionals to provide necessary medical care, illness prevention and harm reduction in otherwise adverse environments and unfavorable conditions.

The present invention uses advanced algorithms and artificial intelligence within a virtual, cloud-based graph platform to track, measure, record, and analyze patient care and activity to produce baseline predictors of emerging needs within various populations. These populations include but are not limited to those who are dispossessed for a variety of reasons, such as employment abroad, or because of economic and financial crises and insecurity. The use of filtering and subjective analysis allows for cataloging, identifying, and driving written and recorded content to patients based upon their patterns of use and care.

Due to more personalized care which focuses on prevention, the platform while creating expanded revenue opportunities for health professionals, moves the patient along the path at urgent care to primary care while decreasing their need to seek more costly paths of medical treatments and ensuring better outcomes through lifestyle changes and greater access to convenient care.

The present invention aims at structuring rapid implementation and long-term support. The present invention may also include an implementation plan that is cloud-based, such as the Vive Private Cloud Environment Setup, wherein a medical concierge feature, such as Vive Concierge, will use the private cloud-based servers for accessing, processing, logging, storage, breaches, and redundancy backup of population health information. This implementation is based on the HIPAA Security Controls Mapping Implementation Playbook.

The platform is being distributed through commercial partners in travel, community health, spa, sports, entertainment, health services, such as in-home testing, insurance industry for health improvement and claim reduction, fitness, residential and planned communities, as well as corporate clients given it improves employee satisfaction and health. As well the platform will be used and promoted by local municipalities such as the city of Los Angeles as well as countries such as Ethiopia given its ability to extend primary care irrespective of geography. Please reference feature list in “invention description.”

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The various embodiments are illustrated by way of example, and not by way of limitation, in the figures of the accompanying drawings. Having thus described the invention in general terms, reference will now be made to the accompanying drawings, which are not necessarily drawn to scale, and wherein:

FIG. 1 is an overview of the present invention for healthcare integration-as-a-service.

FIG. 2 is a flow diagram of the private cloud-based application.

FIG. 3 is a diagram of the end-to-end data flow of the present invention.

FIGS. 4A-B are diagrams of the basic platform architecture of the present invention.

FIG. 5 is a diagram of the integrated data sets of the present invention.

FIGS. 6A-B are flow diagrams of the appointment workflow of the present invention.

FIG. 7A-B are flow diagrams of the appointment and chat workflow of the present invention.

FIG. 8 shows a comparison of the present invention to an existing system.

FIG. 9 is a line diagram illustrating a decentralized network.

FIG. 10 is a line diagram illustrating a distributed network.

FIG. 11 is a flow diagram of the feature release process of the present invention.

FIGS. 12A-B show the various healthcare integration roles and responsibilities of the present invention.

FIGS. 13A-T are renderings of the mobile application interface of the present invention.

### DETAILED DESCRIPTION OF THE PREFERRED EMBODIMENT

The present invention is a multi-functional platform that provides concierge medical practitioners with a turnkey solution by way of a technologically advanced tool, in compliance with the Health Insurance Portability and Accountability Act (“HIPAA”). The platform can integrate with external platforms to facilitates physicians and other health care team members to establish, manage, expand, and market independent physician practices to an unlimited number of patients in a HIPAA compliant and organized manner, while reducing overhead by negating the need for office space.

The platform functions as a system and method of delivering care through a virtualized means with advanced tools of managing, treating, and coordinating care around educating, coaching, and sustaining primary care. The platform incorporates advanced technology, such as graphical and artificial intelligence (“AI”) tools, allowing for the patient to customize care as their needs and those of their family are used to create services that are anticipatory of their unique health conditions as they age or experience changes in their health condition.

The present invention's platform is capable of deployment into underserved and rural regions to expand the availability of basic and advanced care, as well as allowing for lower income communities to access qualified physicians by matching low-income persons to physicians and other health care providers offering pro-bono care and support. The platform decreases the burdens on the emergency rooms and lowers overall per patient healthcare costs by focusing on prevention, supporting sustainable outcomes by reinforcing and using holistic care plans, and expanding job creation and employment opportunities for health professionals and independent physician practices. The platform can be used as a turnkey revenue producing tool that enables concierge care, in office and virtual staff expansion and management, and a HIPAA compliant means of delivering services to patients.

The platform is able to provide health care access to “care deserts” wherein limited access to medical care increases instances of aggravated illness and taxes the public care system. The virtual platform allows physicians and other health care team members to provide voluntary pro-bono care to individuals experiencing displacement, housing insecurity, transitional or vagrant living situations due to economic crises, those who live under itinerant conditions and low-income communities (“LICs”). The platform incorporates the use of the US Treasury Community Development Financial Institution (“CDFI”) mapping system to identify, track, and measure engagement within LICs with health professionals.

The present invention is a customized external Application Programming Interface (“API”) platform that integrates patient data, health care resources and information through mobile online accessibility, search, geolocation, and notification functionality. The API platform facilitates health care professionals to provide necessary medical care, illness prevention and harm reduction in otherwise adverse environments and unfavorable conditions.

The present invention uses advanced algorithms and artificial intelligence within a virtual, cloud-based graph platform to track, measure, record, and analyze patient care and activity to produce baseline predictors of emerging needs within various populations. The use of filtering and subjective analysis allows for cataloging, identifying, and driving written and recorded content to patients based upon their patterns of use and care.

Due to more personalized care which focuses on prevention, the platform while creating expanded revenue opportunities for health professionals, moves the patient along the path at urgent care to primary care while decreasing their need to seek more costly paths of medical treatments and ensuring better outcomes through lifestyle changes and greater access to convenient care.

The present invention aims at structuring rapid implementation and long term support. The present invention may also include an implementation plan that is cloud-based, such as the Vive Private Cloud Environment Setup, wherein a medical concierge feature, such as Vive Concierge, will use the private cloud-based servers for accessing, processing, logging, storage, breaches, and redundancy backup of population health information. This implementation is based on the HIPAA Security Controls Mapping Implementation Playbook.

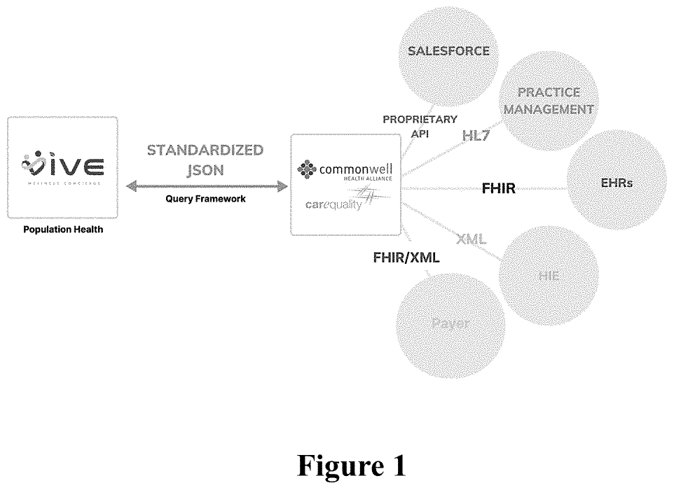

FIG. 1 is an overview of the present invention for healthcare integration-as-a-service. In accordance with the preferred embodiment, the present invention uses technology-enabled information sharing of electronic health records (“EHR”) to build upon existing applications and EHR platforms to establish an interoperable system of coordination where all street medicine providers can access patient health information and coordinate across the delivery system.

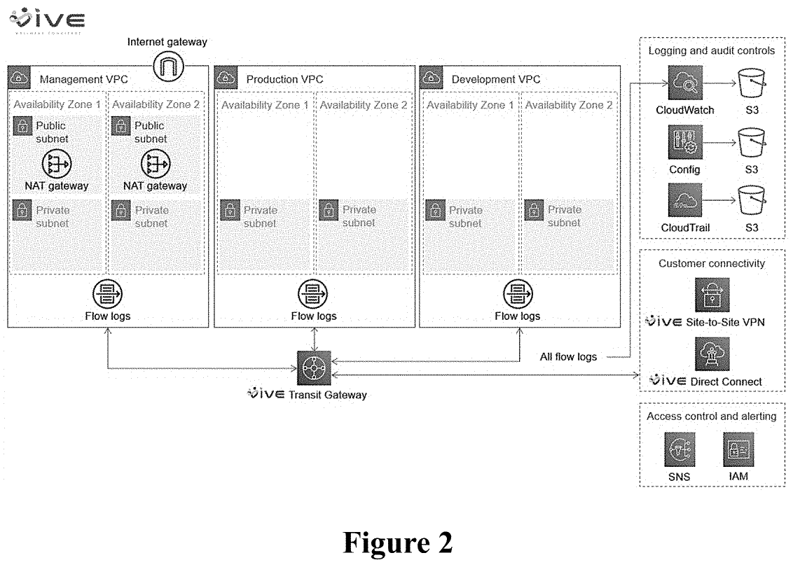

FIG. 2 is a flow diagram of the private cloud-based application. The application consists of virtual private cloud (“VPC”) technology, for an on-demand configurable pool of shared resources allocated within a public cloud environment, providing a certain level of isolation between the different organizations using the resources. The application platform consists of a management VPC, a production VPC, and development VPC, connected via a transit gateway to flow logs, audit controls, customer connectivity, access control and alerting.

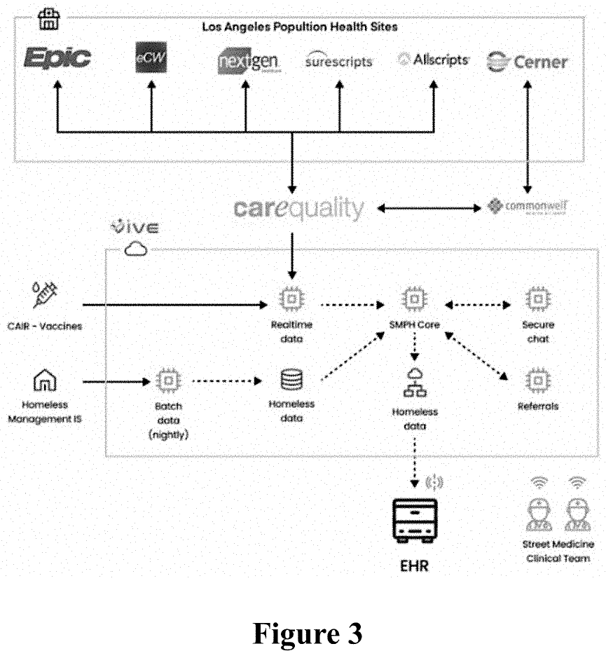

FIG. 3 is a diagram of the end-to-end data flow of the present invention. In accordance with the preferred embodiment, tackling complex problems starts with understanding the data flow and human needs involved. The platform of the present invention can send and receive patient records, including data such as: registration; medication; allergies; lab/radiology; medical history and medication history software (“Med Hx”); vaccinations; clinical notes; Homeless Management Information System (“HMIS”) data. The platform offers real-time coordination and communication, including secure text; provider referrals; and geolocation features.

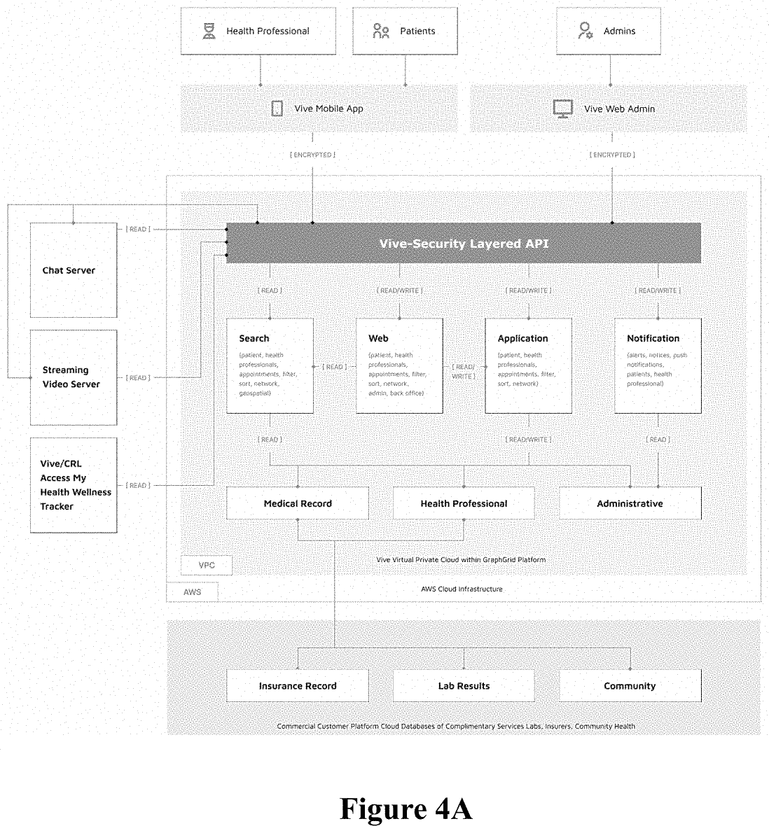

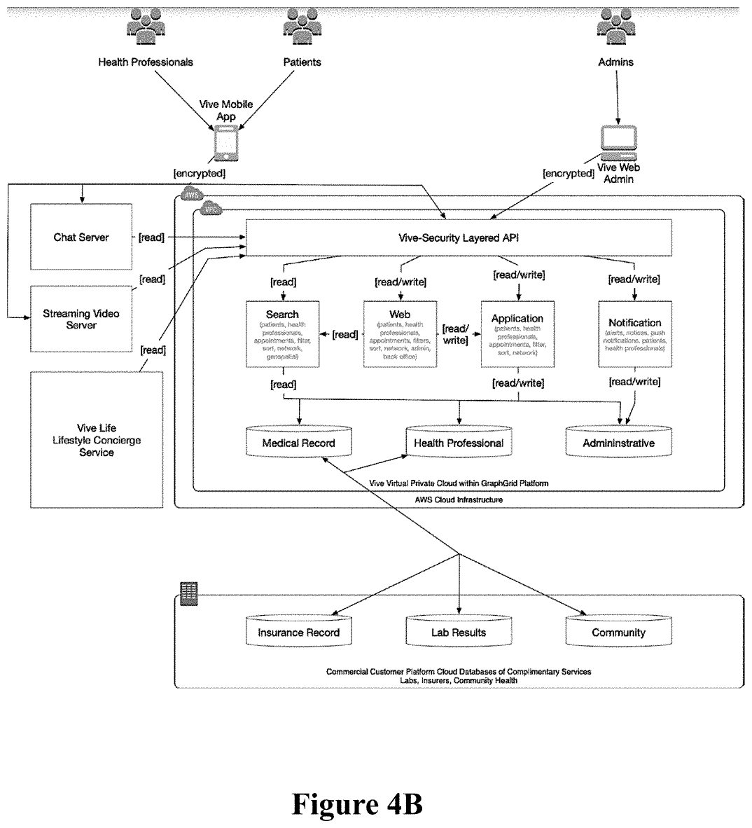

FIGS. 4A-B are diagrams of the basic platform architecture of the present invention. In accordance with the preferred embodiment of the present invention, health professionals and patients can use the mobile application to access the platform through a security layered API. Administrators can also access the platform through a web admin portal. The security layered API provides functions that include search, web, application and notification. Through these functions, health professionals and patients can access medical records, as well as health professional and administrative modules. The API is located on a virtual private cloud (“VPC”) infrastructure. The medical record and health professional modules also allow for access to insurance records, lab results, and the overall community of the platform. The security layered API can also provide health professionals and patients with access to the platform's chat server, streaming video server, and access to a health and wellness feature.

The web feature allows admin, patients, and health professionals to access the search engine for patients, health professionals, and appointments with the option to sort the search results based on insurance network, location, and other filters. It also provides a back office and administrative features for the admin access. The application feature is also accessible by admin, health professionals, and patients and acts as the user interface for each, providing access to patient information, health professional information, appointments, and more. The user interface is tailored to the user, and appears different for health professionals and patients, respectively. Users may choose to enable notifications, which provides the user with alerts, notices, and push notifications for upcoming appointments, messages from patients or health professionals, reminders, special offers, and more. All the data collected such as lab results and insurance records are stored on commercial customer platform cloud databases and may be securely accessed by the respective patient and sent to the patient's preferred providers when applicable.

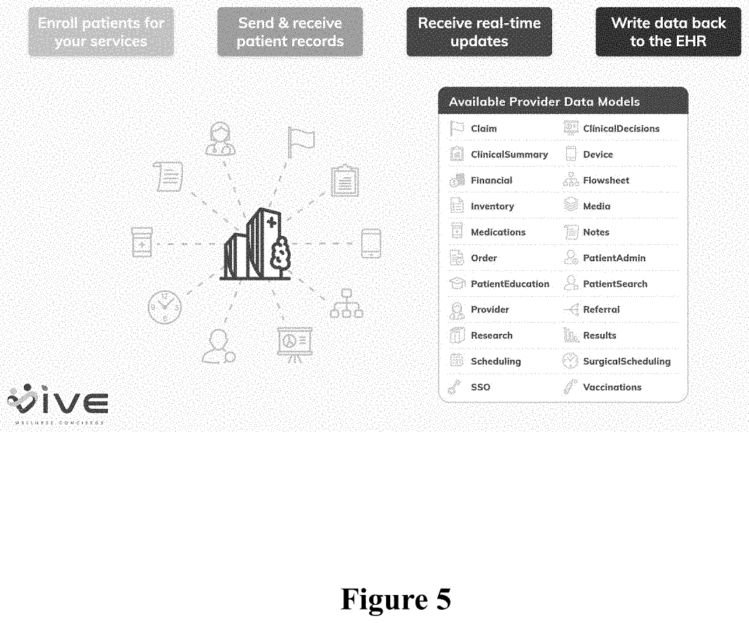

FIG. 5 is a diagram of the integrated data sets of the present invention. In accordance with the preferred embodiment, the platform offers features such as patient enrollment, sending and receiving patient records, real time updates, and writing data to electronic heath records (EHR). The platform also features various provider data models, such as: claims; clinical decisions and summaries; mobile device; financial; flow sheet; inventory; media; medications; notes; orders; patient admin, education and search; provider; referral; research; results; appointment and surgical scheduling; single sign on (“SSO”); and vaccinations.

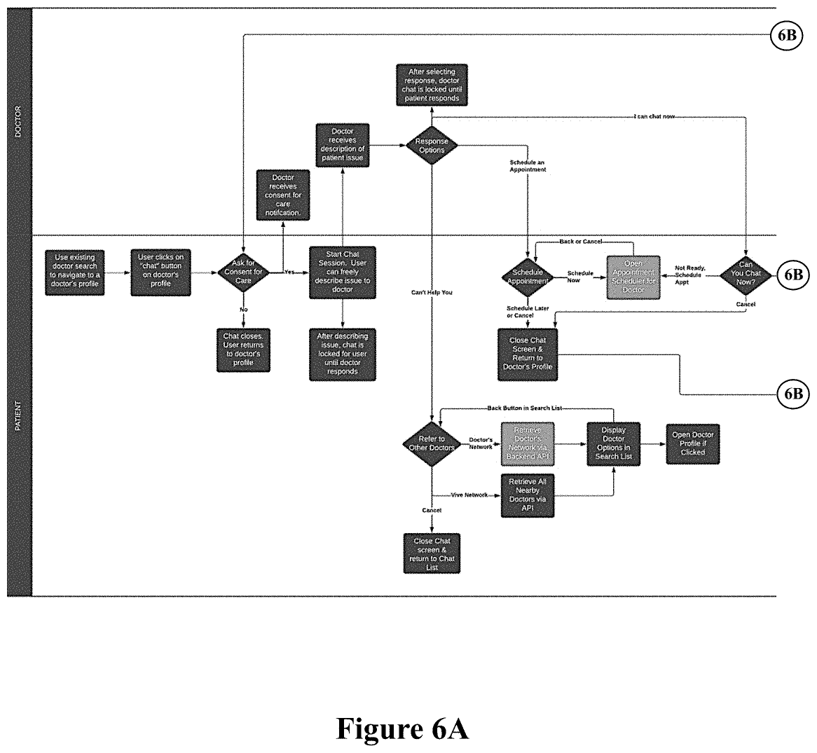

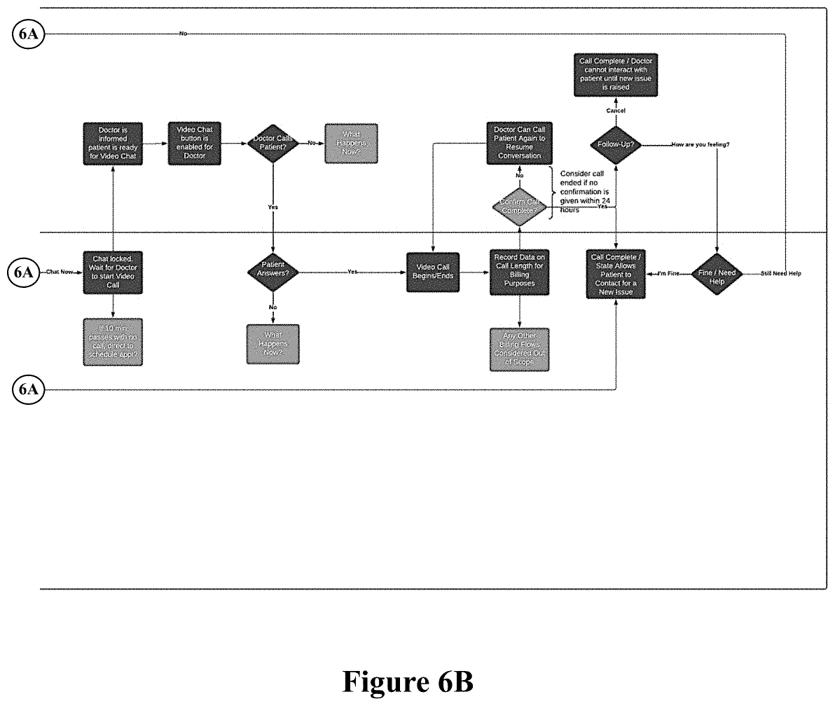

FIGS. 6A-B are flow diagrams of the appointment workflow of the present invention. In accordance with the preferred embodiment, the patient user can use the platform's existing doctor search to navigate to a doctor's profile. The user clicks on the “chat” button on the doctor's profile, which is followed by a verification of consent for care from the patient user. If the patient selects no, the chat function closes and the user is returned to the doctor's profile. If the patient selects yes, the doctor receives a notification of the consent and the chat session can start, wherein the user can freely describe the issue to the doctor. After describing the issue, the chat is locked for the user until the doctor responds. The doctor receives the description of the patient's issue from the chat, and is then able to select from response options. If the doctor cannot help the patient with the specific issue, the doctor can then refer the patient to other doctors within the doctor's network or platform network. The doctor network is retrieved through a backend API, and the doctor options are displayed on a search list, allowing for the patient to select another doctor. The chat is then closed. If the doctor agrees to help the patient, the next step is to schedule an appointment, allowing the patient to schedule now or schedule at a later time. The patient can also chat with the doctor if the doctor is available at that time.

If the doctor is available, the chat is then locked until the doctor starts a video call for a virtual appointment. The doctor is then informed that the patient is ready for a video chat, and the video chat button is enabled. The doctor initiates the video call appointment, and once the patient answers, the appointment officially begins. Data such as the length of the call is recorded for billing purposes. Once the appointment call is complete, the doctor can call the patient again to resume another conversation, or the appointment is considered complete if no follow up is necessary. The patient is then able to contact the doctor if they have a new issue.

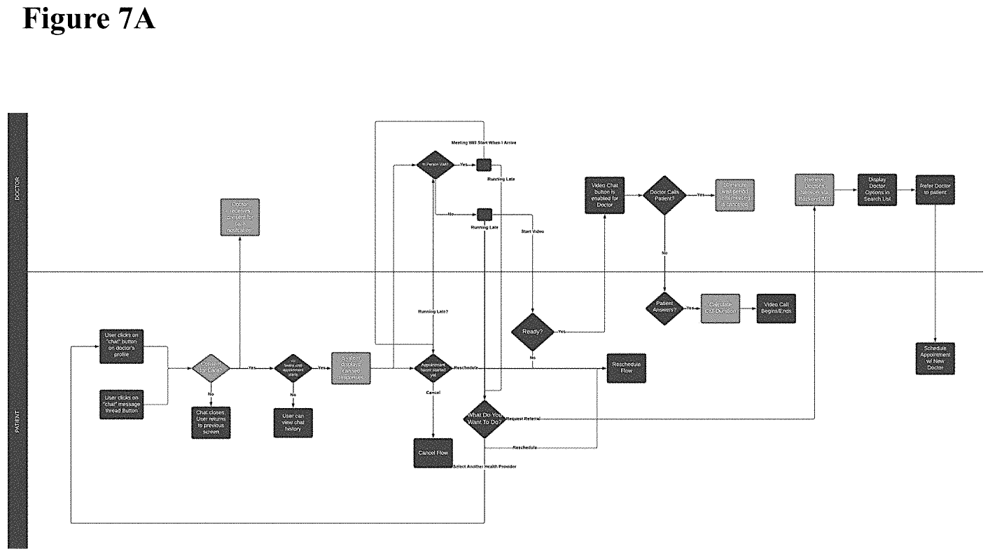

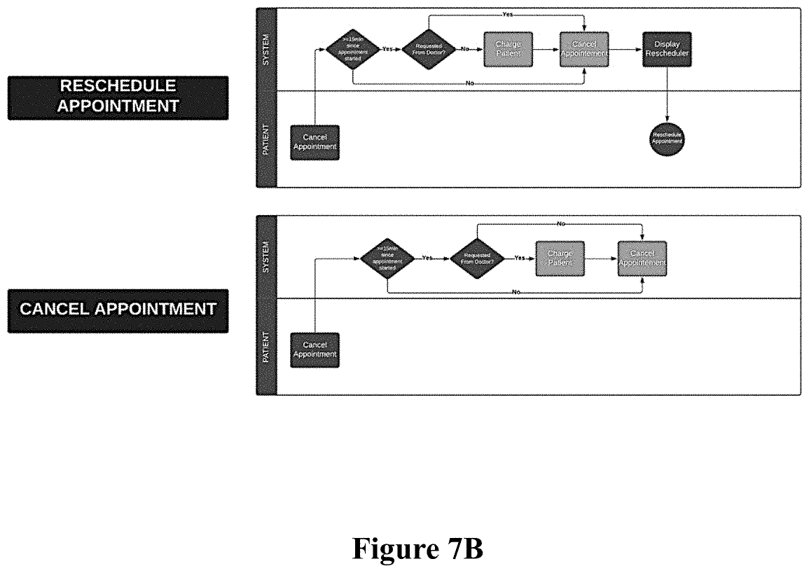

FIG. 7A-B are flow diagrams of the appointment and chat workflow of the present invention. In accordance with the preferred embodiment, the patient user can click on the “chat” button on a doctor's profile, or click on the “chat” message thread button. Once the user gives consent for care, the doctor receives a notification of consent, and the patient is notified that the appointment will start in a specific amount of time, i.e., 5 minutes. The patient is able to cancel the appointment within that time or reschedule if necessary. Once the doctor is ready to start the appointment, a video chat button is enabled and the appointment can begin when the doctor calls the patient. If the doctor cannot help the patient with the specific issue, the doctor can then refer the patient to other doctors within the doctor's network or platform network. The doctor network is retrieved through a backend API, and the doctor options are displayed on a search list, allowing for the patient to select another doctor. The patient can then schedule an appointment with another doctor.

If the patient chooses to cancel the appointment, the patient can be charged and is presented with the rescheduling module to reschedule the appointment. If the doctor has requested to reschedule the appointment, the patent will not be charged and is able to reschedule the appointment through the rescheduling module. The patient or doctor can also cancel the appointment without rescheduling another appointment.

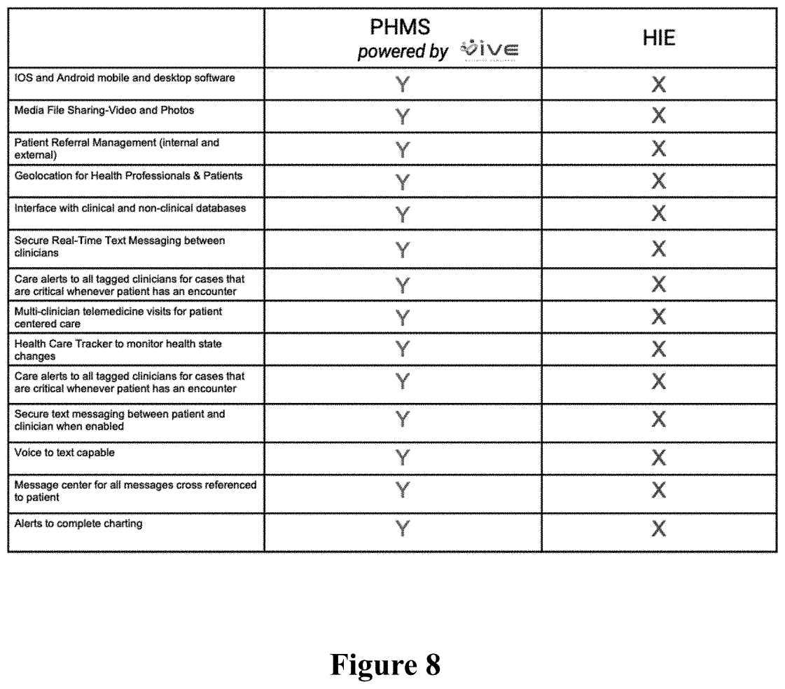

FIG. 8 shows a comparison of the present invention to an existing system, such as the electronic health information exchange (“HIE”). The present invention is a Population Health Management System (“PHMS”) that utilizes a multitude of features not offered by HIE, including but not limited to: IOS and Android mobile and desktop software; media file sharing-video and photos; patient referral management (internal and external); geolocation for health professionals & patients; interface with clinical and non-clinical databases; secure real-time text messaging between clinicians; care alerts to all tagged clinicians for cases that are critical whenever patient has an encounter; multi-clinician telemedicine visits for patient centered care; health care tracker to monitor health state changes; care alerts to all tagged clinicians for cases that are critical whenever patient has an encounter; secure text messaging between patient and clinician when enabled; voice to text capable; message center for all messages cross referenced to patient; and alerts to complete charting.

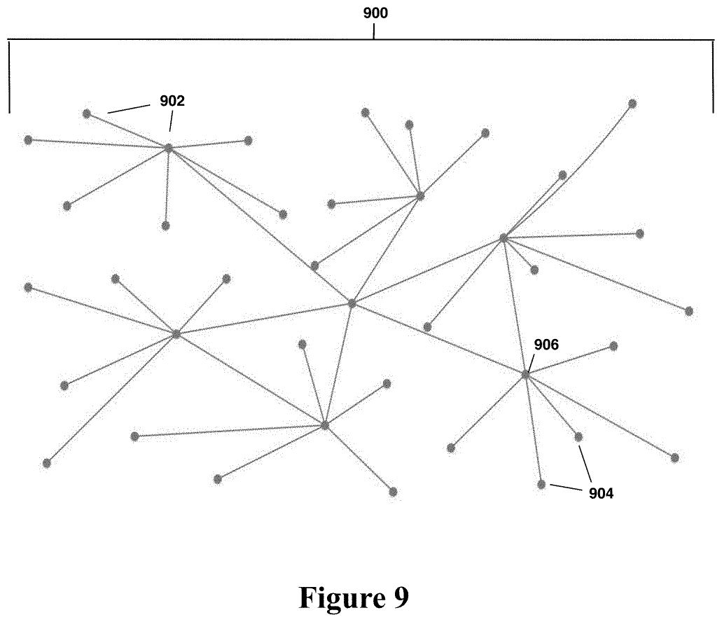

FIG. 9 is a line diagram illustrating a decentralized network. In accordance with the preferred embodiment of the present invention, the specific architecture of the network can be either decentralized or distributed. FIG. 9, generally represented by the numeral 900, provides an illustrative diagram of the decentralized network. FIG. 9 depicts each node with a dot 902 Under this system, each node is connected to at least one other node 904. Only some nodes are connected to more than one node 906.

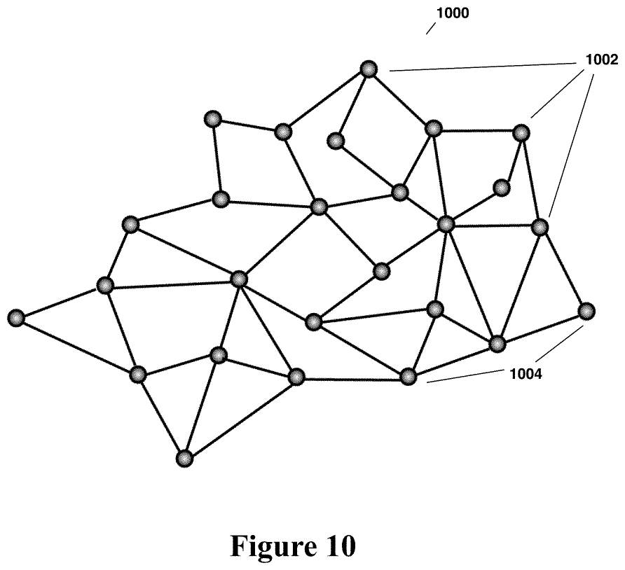

FIG. 10 is a line diagram illustrating a distributed network. For comparison purposes, FIG. 10, which is generally represented by the numeral 1000, illustrates a distributed network. Specifically, the illustration shows the interconnection of each node 1002 in a distributed decentralized network 1000. In accordance with the preferred embodiment of the present invention, each node 1002 in the distributed network 1000 is directly connected to at least two other nodes 1004. This allows each node 1002 to transact with at least one other node 1002 in the network. The present invention can be deployed on a centralized, decentralized, or distributed network.

In one embodiment, each transaction (or a block of transactions) is incorporated, confirmed, verified, included, or otherwise validated into the blockchain via a consensus protocol. Consensus is a dynamic method of reaching agreement regarding any transaction that occurs in a decentralized system. In one embodiment, a distributed hierarchical registry is provided for device discovery and communication. The distributed hierarchical registry comprises a plurality of registry groups at a first level of the hierarchical registry, each registry group comprising a plurality of registry servers. The plurality of registry servers in a registry group provide services comprising receiving client update information from client devices, and responding to client lookup requests from client devices. The plurality of registry servers in each of the plurality of registry groups provide the services using, at least in part, a quorum consensus protocol.

As another example, a method is provided for device discovery and communication using a distributed hierarchical registry. The method comprises broadcasting a request to identify a registry server, receiving a response from a registry server, and sending client update information to the registry server. The registry server is part of a registry group of the distributed hierarchical registry, and the registry group comprises a plurality of registry servers. The registry server updates other registry servers of the registry group with the client update information using, at least in part, a quorum consensus protocol.

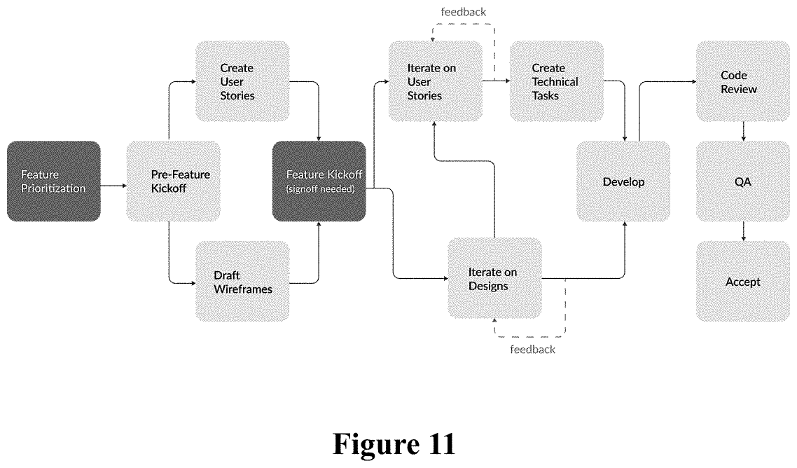

FIG. 11 is a flow diagram of the feature release process of the present invention. In accordance with the preferred embodiment, all application features are subjected to this process before implementation into the platform.

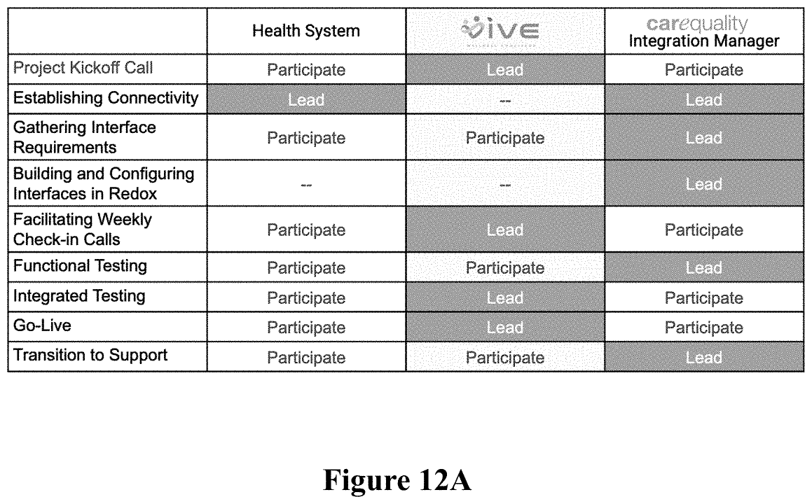

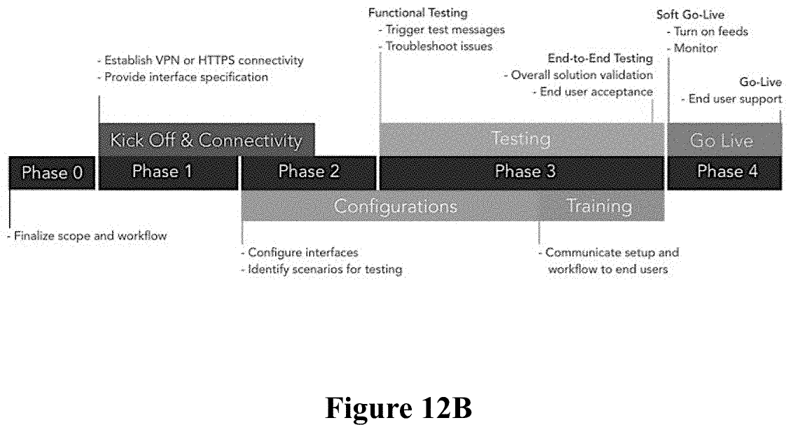

FIGS. 12A-B show the various healthcare integration roles and responsibilities of the present invention. FIG. 6A shows the various healthcare integration roles and responsibilities of the present invention, including the project kickoff call; establishing connectivity; gathering interface requirements; building and configuring interfaces in redox; facilitating weekly check-in calls; functional testing; integrated testing; go-live; and transition to support. FIG. 6B is a diagram of the integration of the present invention, showing the phases necessary to integrate the platform of the present invention to existing platform structures such as CareQuality.

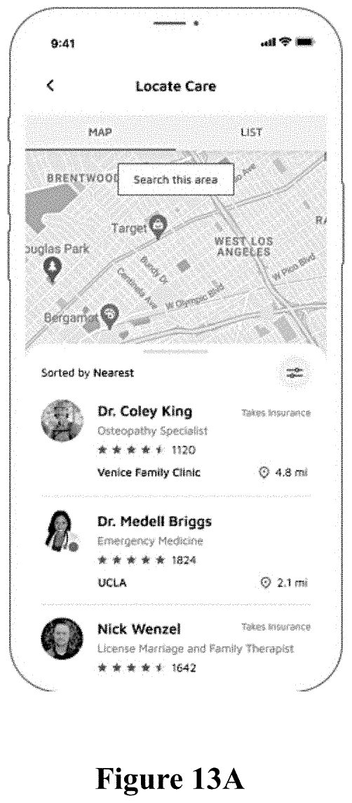

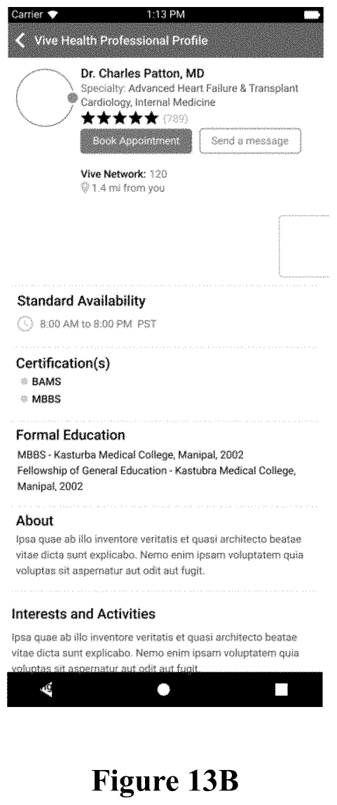

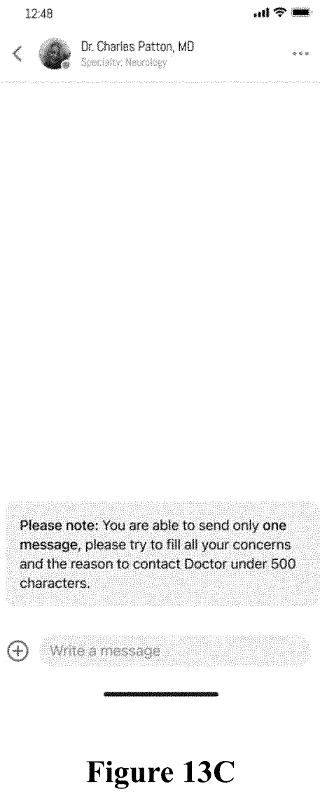

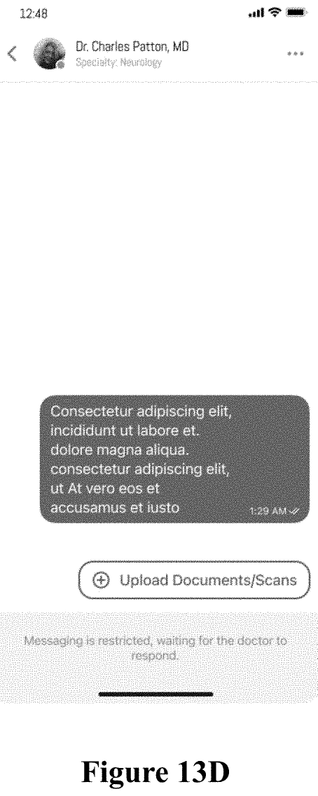

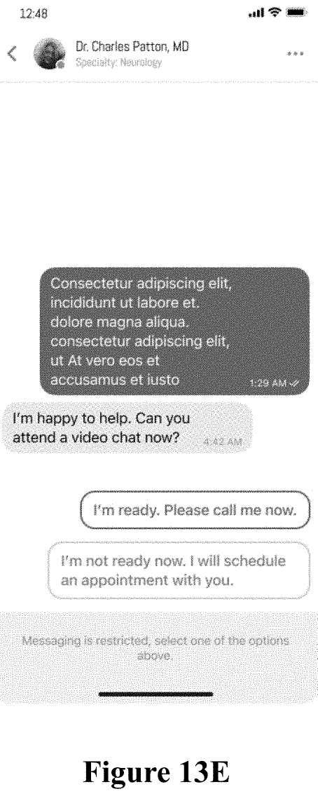

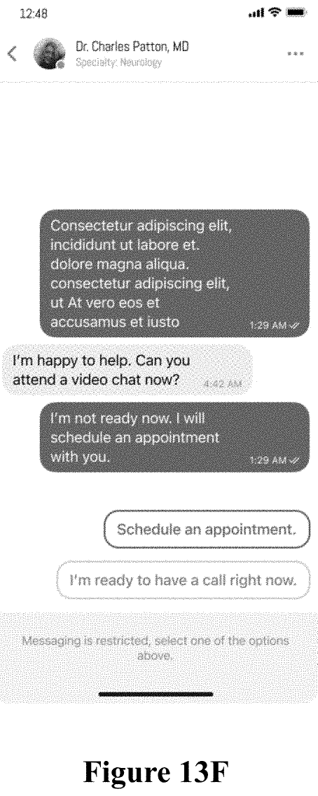

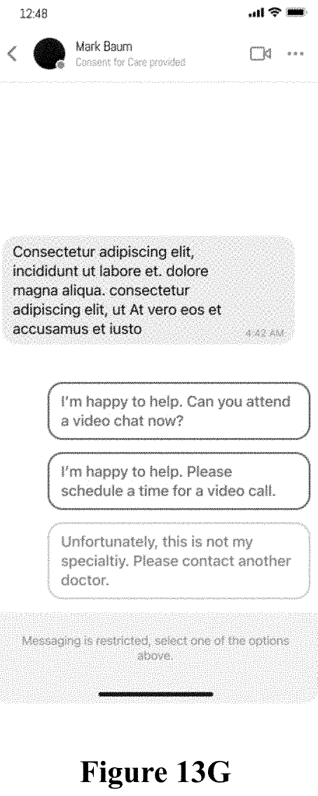

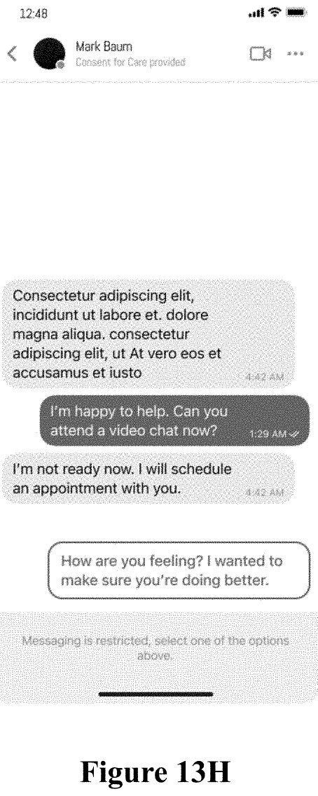

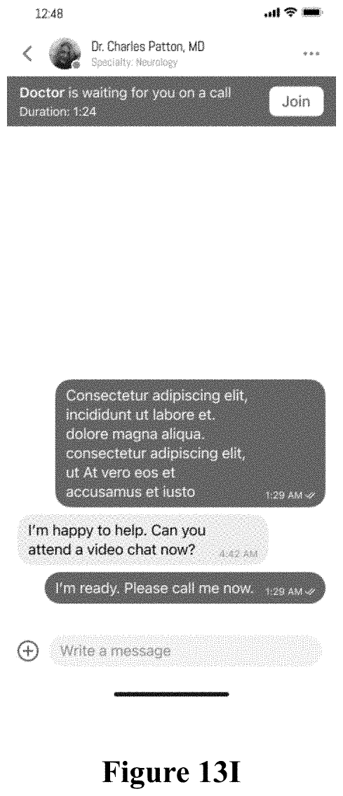

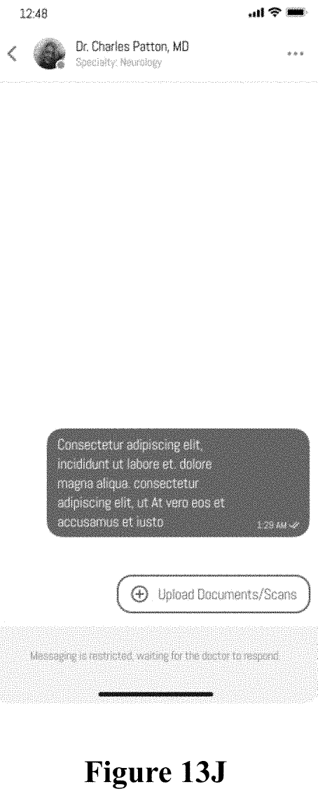

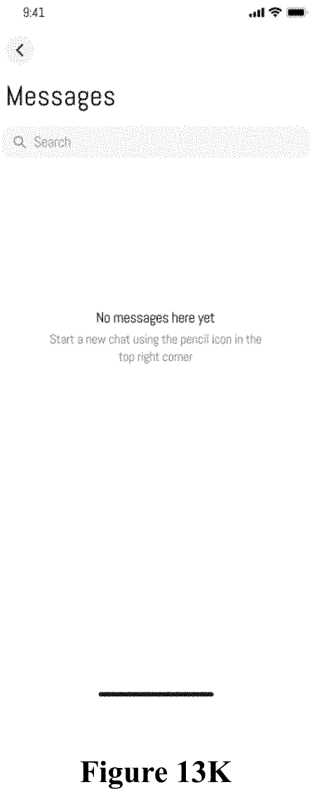

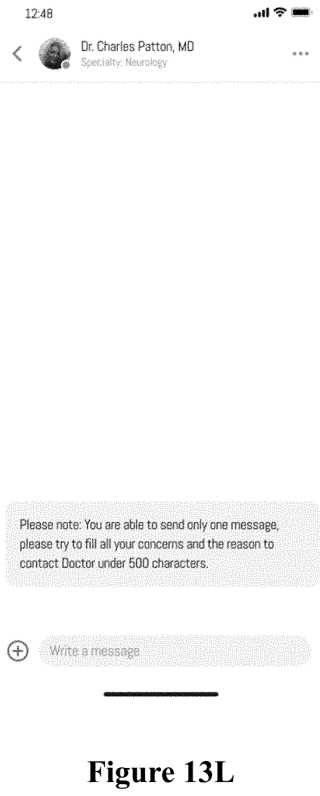

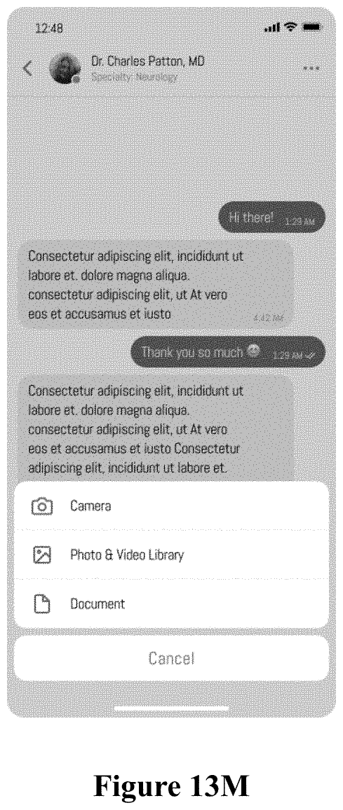

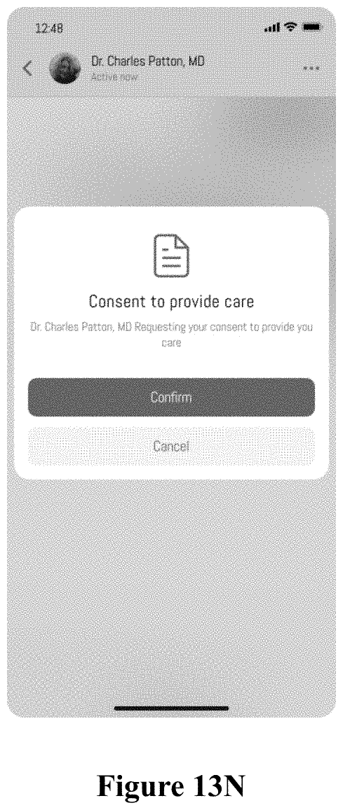

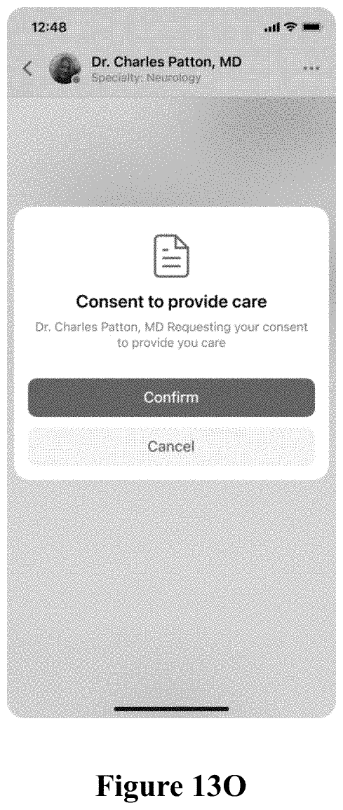

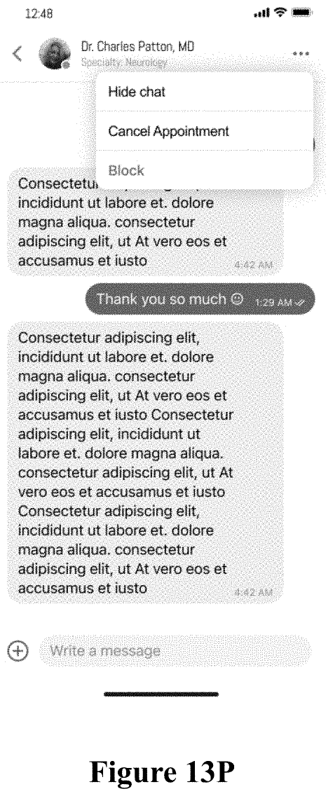

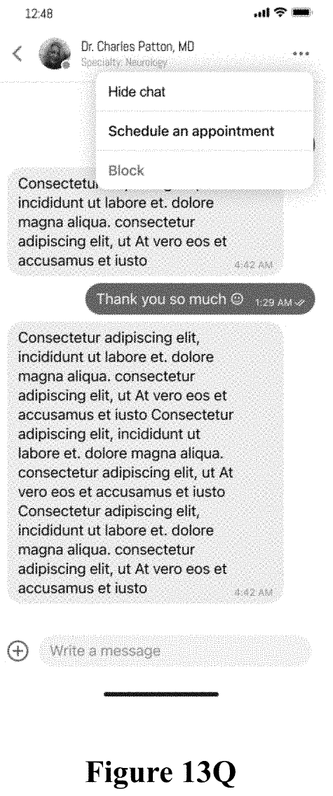

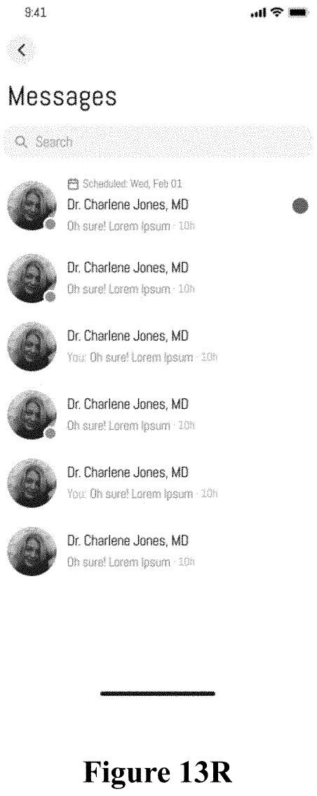

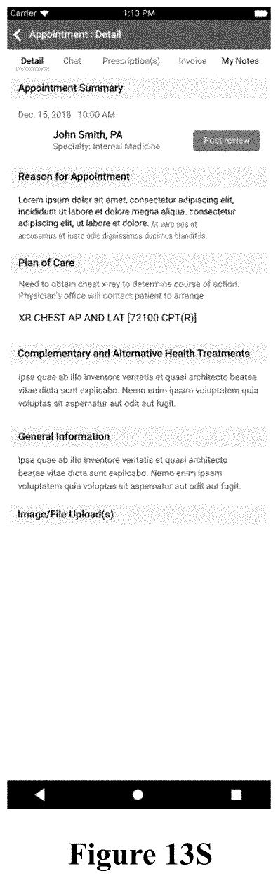

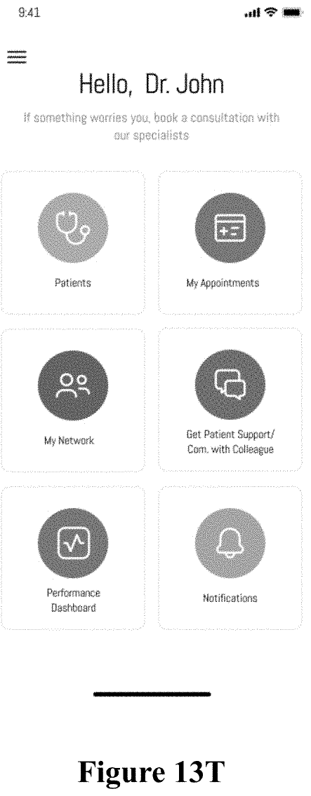

FIGS. 13A-T are renderings of the mobile application interface of the present invention. In accordance with the preferred embodiment of the present invention, the platform can be accessed through a mobile application, which provides geolocation and provider search functionality, as shown in FIG. 13A. Providers can be filtered using specific criteria that allows the patient to filter the type of provider for their healthcare needs based on factors such as geolocation and proximity. FIG. 13B shows a rendering of the doctor profile through the mobile interface. Through the doctor profile page, the patient user can book an appointment or send a message to the doctor. The profile page also shows the doctor's standard availability, certifications, education, and other information about the doctor.

FIGS. 13C-R are renderings of the messaging interface of the mobile application. In accordance with the preferred embodiment of the present invention, patients and health providers can communicate securely via the live chat feature. Messages are saved for future reference unless the user chooses to delete old messages. When a patient or health provider is currently active, they may choose to display their status as “active” which is represented by a green dot adjacent to their icon image. Users may also search for conversations to quickly locate previous messages by either searching keywords or names. If a patient has scheduled an appointment with a health professional, the appointment date will appear above their most recent chat conversation with the health professional. FIG. 13S is a rendering of the appointment detail of the mobile application including the date and time of the appointment, the doctor, the reason for the appointment, the plan of care, options for alternative treatments and other relevant health information. FIG. 13T is a rendering of the doctor's main interface of the mobile application. The main screen allows for doctors to access patient data, appointment information, the doctor's network, communication with colleagues, a performance dashboard and notifications.

The present invention may be accessed through an application, such as one that can be downloaded to a mobile device. This application can incorporate various functions, including but not limited to online web access, a search function, administrative functions, health professional functions, and a patient portal with notification capability. The online access and search functionality may include a geolocation feature, locating all physicians within a specified distance that are immediately available for walk-in to an office appointment and for concierge visits, proximity of the patient to all urgent care centers across the US to expedites patient access, as well as coordinate the delivery of services for a mobile medical team (for example, Vive Van management) for community wellness and prevention engagement. This is coordinated engagement within low-income communities (“LICs”). Patients can also use the application to search for a physician and other health care provider profiles for care locally, nationally, and internationally. The application platform incorporates geolocation functions to provide clinical point-of-care (“POC”), wherein providers deliver healthcare treatment, products, and services to patients at the time of care. The platform integrates crowdsourced data to deliver medical services and to those in need, at the “point of service” (“POS”). The application's geolocation functionality may also be used for patient intake procedures

The application's administrative functionality facilitates a unique deployment and use of nurse practitioners that can perform most medical functions on the virtual platform and on concierge visits, to diminish costs to patients for care and facilitate expanded physician supervision through administrative oversight managed through the platform. The application allows for advanced background checks and verification of physicians and other health care team members and allows for physicians and other health care team members to be tracked to a patient's location for security and transparency with updated estimated time of arrival (“ETA”) for the patients. The platform can also display average visit times for providers on both the patient side and provider side.

The application's health professional function facilitates patient care and multi-practitioner coordination through a virtual platform to mitigate and remove the need for an in-office physician and other health care team member appointments. The referral process is simplified and streamlined from one provider to the next. Registered nurses can support the patient as health coaches and have platform support that aggregates medical notes with the existing medical record. This is supportive of the plan of care and is unique to each patient and dramatically increases intended outcomes. Support staff from private practices can be granted access by the primary health care provider to work as a team and open communication to save time for the care of the patient. Notes can be stored and shared across the platform for continuity of care.

Physicians and other health care team members can use the application to prescribe standard pharmaceutical medications and track fills to ensure fulfillment was successful. As well, the platform can track compliance of taking the medication. Physicians and other health care team members can also recommend and prescribe holistic medications and treatments to reduce costs to patients and encourage prevention, thereby lowering patient medical costs and reducing side-effects. Physicians and other health care team members can share referenced education material for the patient for later reference in an organized manner. This is inclusive of alternative protocols which will support the best holistic care. Case management, coordination and support can be managed through the application between patients. The application may also include a patient advocate support tool for health providers, patients, and caregivers involved in the patient's care.

The application platform may also integrate technology such as artificial intelligence to intuitively make recommendations based off a patient complaint. The application can incorporate real time charting, as well as dictation and transcribing technology for ease of use. The application may also include filtering capabilities to filter patients into certain groups for easy communications, for example, diabetics. The provider can then send specific information to diabetic patients in that specific group to facilitate in patient health management.

The application provides software license revenue for practitioners, thus increasing revenues for independent physician practices and off-setting costs. This allows for physicians and other health care team members to be able to monetize and rate their health care networks through the ability to establish and manage their own “ranking system” of to whom to flow their referrals through the automated platform resulting in quality assurance checks within a virtual ecosystem for supervising physicians. The application provides an advanced rating system for providers as rated by patients, serving as an additional marketing tool for physicians and other health care team members. The provider reviews aid in supervision of practitioners if there is a private review system in place.

The application may also be used as a tool to aid in medical compliance, organization, and best practices, such as healthcare reminders and alerts that are sent to both providers and patients, and notifications to providers for license expiration and renewal reminders. Providers may also be able to upload their practice consent forms and other compliance related documents.

The application can be used by patients to facilitate the ordering of lab work, tracking, and presenting in home blood tests and intravenous (“IV”) services across a multitude of panels through a virtual platform. The application can facilitate, track, and arrange for band delivery of products as well as the shipment of wellness products and pharmaceutical medications. The application facilitates prevention of illness through measures to mitigate claims and losses, as well as prevention of illness and critical therapy needs for non-emergent patients in need of therapeutic care. The patient can refer to previous encounters and read up on their prescriptions and protocols within their health record profile. The application platform can also accommodate data entry such a blood sugar levels, blood pressure readings. The platform may also incorporate features such as third person consent and consent for minors for healthcare treatment.

The application affords patient convenience and expands options by allowing for virtual medical records to be easily stored and accessed through the application, permitting a greater continuity of care for both patient convenience and safety. The platform can allow for health record access to patients, resulting in full healthcare transparency. The application provides a means for virtual visits inclusive of medical charting, historical medical record—through electronic medical record (“EMR”) access, and advanced health profile and treatments through a virtual platform. The patient's health record is inclusive of holistic modalities, treatments, and plan of care. The application can also track and measure patient health changes and vitals through graphical analysis.

The application may also function as a research and clinical trial communications portal. Collections of data can be aggregated, stored, and accessed, such as labs, prescriptions, and other holistic alternatives. As data is collected over time, this can be a very rich information source for certain industries. The application can generate digital health questionnaires as a means of aggregating data. Certain applicable advertisements may also be incorporated into the platform, as well as an option for patients to opt in or out.

Medical doctors can use the platform to publish papers and other works, promoting and creating standing protocols for holistic and western treatments that can be shared globally. Medical doctors may use the application as a means of holding lectures and seminars, broadcasting live and streaming globally. Continuing education courses may also be offered through the platform, providing continuing education for health professional management. Providers can pull from an integrative library, such as relevant documents and journals to share with college students, etc. The platform may also incorporate programs such as a health coach program for Holistic registered nurses (“RNs”). Typically, coaches can be defined as non-medical personnel with certificates in health coaching. RNs can speak on healthcare treatment and the disease process, serving as teachers and instructors through the platform

The present invention can mimic the experience of stepping into a medical center for patients, as well as the “gamification” of the physician experience. The application can offer a medical concierge practice service, with elite care access to elite practice groups, admitting privileges, upgrades, and access to concierge services such as Vive life or Vive practice group, which provides access to highly specialized medical providers. Payment for medical services and healthcare related costs can also be processed through the platform, as well as payment by gift cards such as the Vive gift card, etc.

The platform can also include a multitude of optional services in the health and wellness space, including but not limited to: specialty consulting for lab work; anti-aging; medical cannabis or medical marijuana prescribers and resources; rehabilitation and detox resources; stem cell therapy; nutritional supplement resources and e-commerce; veterinary services and resources; community impact resources, such as information related to community meetings and outpatient recovery groups; volunteering opportunities and donating hours or pro-bono opportunities; and pilot programs such as “minute clinics” in retail pharmacies that also offer technician support and various specialists.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

What is claimed is:

1. A computer-implemented system for providing healthcare management for patients and healthcare professionals, the system comprising:
a plurality of computer processors;
a plurality of virtual private cloud platforms;
a memory coupled to said plurality of computer processors storing instructions that, when executed by said plurality of computer processors, cause the system to:
securely receive, from at least one third-party user, via at least one third-party user device, information related to said user's health including history of illness, weight, height, lifestyle habits, current symptoms, and emotional wellness;
securely redirect said information related to said at least one third-party user's health to said plurality of virtual private cloud platforms for data comparisons and analysis;
analyze, on both an individual and a population level, by way of machine-learning algorithms, said information related to said at least one third-party user's health to produce baseline predictors of emerging needs for said user;
compare by way of machine-learning algorithms, said user health data with other collected data from surrounding populations to produce baseline predictors of emerging needs for said surrounding populations;
redirect, based on said machine-learning analysis of said information related to said at least one third-party user's health, said user data to at least one healthcare professional interface; and
securely facilitate communication between said at least one third-party user device and at least one third-party healthcare professional device.

2. The system of claim 1, wherein the system has access to electronic health records associated with said user including lab results, insurance records, and financial information.

3. The system of claim 1, wherein said plurality of virtual private cloud platforms includes a management virtual private cloud, a production virtual private cloud, and a development virtual private cloud all of which are connected via a transit gateway to flow logs, audit controls, customer connectivity, access control, and alerting.

4. The system of claim 1, wherein the system provides real-time updates to the user.

5. The system of claim 1, wherein the system is implemented as a mobile application on said at least one third-party user device and at least one third-party healthcare provider device.

6. The system of claim 5, wherein said user and said healthcare professional communicate via a secure private chat server enabled on said mobile application.

7. The system of claim 5, wherein said user and said healthcare professional communicate via a secure streaming video server enabled on said mobile application.

8. A computerized method for providing healthcare management for patients and healthcare professionals, the method comprising:
receiving, from at least one third-party user, via at least one third-party user device, information related to said at least one third-party user's health;
securely redirecting said information related to said at least one third-party user's health to a plurality of private virtual cloud platforms for data analysis and comparison;
analyzing, via a machine-learning algorithm, said information related to said at least one third-party user's health in order to produce baseline predictors;
comparing, via a machine-learning algorithm, said information related to said at least one third-party user's health with data collected from surrounding populations in order to produce baseline predictors for said surrounding populations;
redirecting, based on said machine-learning analysis of said information related to said at least one third-party user's health, said user data to at least one healthcare professional interface; and
securely facilitating communication between said at least one third-party user device and at least one third-party healthcare professional device.

9. The method of claim 8, wherein electronic health records associated with said user are accessed, including lab results, insurance records, and financial information.

10. The method of claim 8, wherein real-time updates are provided to said user.

11. The method of claim 8, wherein said user may access a user interface via a mobile application on said at least one third-party user device.

12. The method of claim 8, wherein said user is provided with a plurality of recommended healthcare strategies to address their current health concerns, said plurality of recommended healthcare strategies being developed via machine-learning algorithm designed to receive information from said private virtual cloud platforms.

13. The method of claim 11, wherein said user and said healthcare professional communicate via a secure private chat server enabled on said mobile application.

14. The method of claim 11, wherein said user and said healthcare professional communicate via a secure streaming video server enabled on said mobile application.

15. A computerized system for facilitating healthcare management, the system comprising:
a plurality of computer processors;
a secure network including a plurality of private virtual cloud platforms;
at least one third-party user device;
at least one third-party healthcare professional device;
a memory coupled to said plurality of computer processors storing instructions that, when executed by said plurality of computer processors, cause the system to:
predict, based on information provided by at least one third-part user, a current health concern pertaining to said at least one third-party user;
determine, via a machine-learning algorithm, additional information needed to confirm said predicted health concern;
request, from said at least one third-party user, additional information such as lab work and previous medical records;
securely redirect all information received from said at least one third-party user to at least one third-party healthcare professional associated with the system;
connect said at least one third-party user with said at least one third-party healthcare professional via a secure user interface;
facilitate communication between said at least one third-party user and said at least one healthcare professional; and
develop, based on information provided by said at least one third-party healthcare professional regarding said at least one third-party user, a unique treatment plan to provide care to said at least one third-party user.

16. The system of claim 15, wherein said user interface is a mobile application accessible by said at least one third-party user and said at least one third-party healthcare professional.

17. The system of claim 15, wherein the system provides said at least one third-party user with recommended healthcare professionals associated with the system based on machine-learning analysis of said at least one third-party user's insurance records, financial status, and specific health concerns.

18. The system of claim 15, wherein the system is integrated with a plurality of pre-existing third-party platforms wherein said at least one third-party user may be redirected from said plurality of pre-existing third-party platforms to said user interface.

19. The system of claim 15, wherein said at least one third-party user is able to customize their care plan and create additional family care plans.

20. The system of claim 15, wherein sub-licensing structures are built into the system in order to improve physician profitability and establish growth of virtual practices.
